(19) United States
(12) Patent Application Publication
(10) Pub. No.: US 2009/0112310 A1
(43) Pub. Date: Apr. 30, 2009

(54) Nanoporous Drug Release Structure for Drug Elute Instruments and the Preparation Method Thereof

(75) Inventor: Yuxin Zhang, Beijing (CN)

Correspondence Address: CONNOLLY BOVE LODGE & HUTZ, LLP, P O BOX 2207, WILMINGTON, DE, 19899, US

(73) Assignee: Lepu Medicql Technology (Beijing) Co., Ltd., Beijing (CN)

(21) Appl. No.: 12/224,588

(22) PCT Filed: Apr. 5, 2007
(86) PCT No.: PCT/CN2007/001109

§ 371 Date: Aug. 28, 2008

(30) Foreign Application Priority Data
Dec. 14, 2006 (CN) 200610168125.0

(51) Int. Cl. A61F 2/44

(52) U.S. Cl. 623/1.42; 623/1.15; 623/1.44; 623/1.39

(57) ABSTRACT

The present invention relates to a nanoporous configuration for drug release used in a drug-eluting device and its preparation, employing acid corrosion or anode oxidation to prepare pores, or employing acid corrosion to prepare pores firstly, then employing anode oxidation or micro-arc oxidation combined with micro-arc nitridation to prepare single sized or two sized or multiple sized nanopores, as well as a uniform size distributed or two or more nonuniform size distributed in pore diameter or pore depth h nanopores on the raw material of device body directly. The preparation process includes: \textcircled{1} Pre-treating the surface of the device body, \textcircled{2} Preparing pore, \textcircled{3} Post-treating the surface of the device body, \textcircled{4}preparing drug, \textcircled{5} Spraying drug etc. The nanoporous configuration lowers the risk of forming thrombus after the drug-delivery device with polymer carrier is implanted into the tissue. The device also controls the release rate of drug efficiently and lowers the incidence of restenosis significantly.

### TECHNICAL FIELD

The present invention relates to a nanoporous configuration for drug release used in drug-eluting device and its preparation.

### BACKGROUND

Drug-eluting device includes various medical devices needing drug release such as vascular stent, duct, guidewire, cardiac pacemaker, cardiac valve, surgical implant material and implanted hard tissue. The vascular stent is a wire metal mesh tube used to prop open for a natural conduit of the body. Stainless steel, titanium alloy, cobalt alloy, and nickel-titanium shape memory alloy etc. can be used to produce stents. The vascular stent is a main method for interventional therapy on cardiovascular and peripheral vascular occlusion diseases. The feature of the stent is that it can moved into the target position through the small tube, and swell to the predetermined diameter after release to hold the conduit open and maintain the conduit unobstructed. Vascular stents can be divided into bare stents, drug-eluting stents, polymer coated stents, metal coated stents, radioactive stents and transluminal stents based on the state of surface. Bare stents were used first. However, stents are heterologous materials for blood vessel and other vessels in the body, the placement of stents will stimulate the inner membrane of blood vessel and cause reactive hyperplasia and restenosis. The incidence of restenosis reaches to 30%-35%. The blood vessel with long-distance disease area or with a smaller diameter will suffer restenosis easier. In order to avoid restenosis, radioactive stents and drug-eluting stents became to be in used. Moreover, drug-eluting stents are well known as the most effective vascular stents resistant to restenosis in interventional therapy for coronary heart disease.

As shown in FIG. 1, the present drug-eluting stents employ polymer as the carrier to deliver drug and control its release, which was prepared by combining active drug coating with polymer on the partial or whole surface of bare stents. In FIG. 1, stent body 10 was coated with a polymer layer 30 containing active drug 70, another polymer layer 30a was coated over polymer layer 30. This kind of stent coated with polymer layer can reduce the incidence of restenosis to less than 10%. However, after this drug-coated stent was implanted into the body, the total of drug will decrease and the concentrate of the polymer will relatively increase, which may result in thrombus. Moreover, the procedure of preparation for this kind of stent is complicated, the production period is long and the cost is high.

As shown in FIG. 2, in order to resolve the above problems, the present drug carrier systems often use a laser to cut pores on the body of the drug-eluting device or use other drug storage systems. Drug is loaded in these pores or other drug storage systems. The size of the smallest pore is micron level, and the pores even can be seen by eye; in FIG. 2, pores 50 used to store drug 70 for restenosis resistance are embedded in device body with uniform distribution. The smallest size of these pores 50 is micrometer level, or even can be seen by eye. Although these micron level or bigger pores 50 can take advantage of loading a big dose of drug 70, the rapid release of drug, the reducing property of supporting force or other physical property of the stent body will come out hereafter.

### DISCLOSURE OF THE INVENTION

### Technical Problem

The purpose of the present invention is to provide a nanoporous configuration for drug release used in drug-eluting devices to overcome the objections in the prior art. This configuration lowers the risk of forming thrombus after the drug-deliver device with polymer carrier is implanted into the tissue. The device also controls the release rate of drug efficiently and lowers the incidence of the restenosis significantly.

It is another object of the present invention to provide a preparation method for nanoporous configuration for drug release used in drug-eluting devices with a simple process and short production period.

### Technical Solution

To achieve the above objects, the present invention employs the technical solutions as follows:

The nanoporous configuration for drug release used in drug-eluting device of the present invention comprises a device body, some pores on the device body and active drugs existed in these pores or adhered to the device body surface. In which, said pores with single size or double sizes or multiple sizes are nano pores. It is said that n nanopores are in a uniform size or in two or more different sizes of diameter and depth on statistical average.

The average value of the diameters of the said nanopores (d) and the depths of said pores (h) is 1 nm-500 μm.

The device body includes a membrane on the external surface.

The single sized pores are any one of the uniform sized nanopores, large nanopores, small nanopores, deep nanopores and shallow nanopores.

The two sized pores include large nanopores and small nanopores with different diameters, or deep nanopores and shallow nanopores with different depths, wherein the active drugs are loaded.

The said multiple nanopores include three or more large nanopores with different diameters and depths, small nanopores with different diameters and depths, deep nanopores and shallow nanopores with different diameters and depths, wherein the active drugs are loaded.

The uniform sized nanopores, such as large nanopores, small nanopores, deep nanopores and shallow nanopores are open pores, half-open pores, closed pores, independent pores, interconnected pores, inter-embedded pores or nested pores or small pores existing in big pores.

The active drug existing in nanopores or adhered to surface of device body includes one or more substances such as a pharmacotherapy agent, vector for gene therapy, and bioactive substance.

The said pharmacotherapy agent includes one or more substance selected from: heparin, aspirin, hirudin, colchicine, antiplatelet GPIIb/IIa receptor antagonist, Methotrexate, purine, miazine, alkaloid and Epothilone, Tripterygium Wilfordii series compound, antibotics, hormone, antibody drug for cancer treatment, cyclosporin, tacrolimus (FK506) and its homologues, 15-deoxyspergualin, Mycophenolate Mofetil (MMF), Rapamycin and its derivatives, FR 900520, FR 900523, NK 86-1086, daclizumab, valeramide (depsidomycin), kanglemycin C, spergualin, 25c(prodigiosin25-c), tranilast, myriocin, FR 651814, SDZ214-104, cyclosporinC, bredinin, mycophenolic acid, Brefeldin A, WS9482, glucocorticosteroid, tirofiban, abciximab, eptifibatide, paclitaxel, actinomycin-D, As₂O₃, 17 β-estradiol.

The vector for gene therapy includes one or more substance selected from: cell, virus, DNA, RNA, virus vector, and non-virus vector.

The bioactive substance includes one or more substances selected from: cell, yeast, bacteria, protein, peptide and hormone.

The device body includes stents, duct, guidewire, cardiac pacemaker, cardiac valve, surgical implant material, implanted hard tissue, and nonmetal medical devices employed ceramic, organic polymer, inorganics, metal oxide as basic material; The said stent is balloon expended stent, self expand stent, vascular stent, non-vascular stent, or stent employing medical stainless steel with good biocompatibility, nickel-titanium shape memory alloy, cobalt alloy, pure titanium, titanium alloy and tantalum, titanium alloy, and gold as basic material, or wire braided, pipe laser cutting, mould casting, and welding stent.

The preparation method for nonporous configuration of the present invention for drug release used in drug-eluting devices, comprises the following steps:
\textcircled{1} Pre-treating the surface of the device body;
\textcircled{2} Preparing pore a, b; this step includes preparing pores by acid corrosion or anode oxidation to make single sized nano pores directly on raw material of device body (10); or firstly making single sized nano pores (50) by acid corrosion on material of device body (10), then making multiple sized nanopore (50) by anode oxidation or micro-arc oxidation combined with micro-arc nitridation.
\textcircled{3} Post-treating the surface of the device body;
\textcircled{4} Preparing drug: preparing 0.01-10% (wt.) active drug (70) in organic solution, dissolved completely; the ratio of the active drug (70) to organic solution is 1:10-1:10000 by weight; and
\textcircled{5} Spraying drug: fixing the device body on spraying machine, and spraying the above prepared active drug (70) on the body material uniformly.

Preferably, the procedure of preparing pores by acid corrosion in \textcircled{2} Includes immersing the device body in a corrosion solution between 0 and 100° C., wherein the corrosion solution is preferred to hydrochloric acid with concentration of 1-38%, or mixed acid solution with 1-30% hydrochloric acid and 1-98% sulfuric acid, or 1-30% hydrofluoric acid, or the above three acids mixed in any concentration. The corrosion time was limited from 1 min to 480 h, thereafter the single sized nano pores are formed.

Preferably, the procedure of anode oxidation in step \textcircled{2} employing the device body material as anode connecting to a positive electrode of pulsed power, titanium flake as cathode connecting to the negative electrode of pulsed power, putting the stent and titanium flake into hydrochloric acid solution synchronously, wherein the electrolyte is preferably hydrochloric acid with concentration of 1-38% or sulfuric acid with concentration of 1-98%, setting the electric current at 0.01-0.1 A, frequency at 25-3000 Hz, time at 1-20 min, and preparing complex nano pores on the body material surface.

Preferably, step \textcircled{1} employs acetone or ethanol solution to clean out the impurities on the device body surface by using ultrasonic waves and then letting it dry.

Preferably, step \textcircled{3} is that the clean device body treated by the above step is washed by acetone and then distilled water under ultrasonic conditions, dried in a dryer or the clean device body is immersed in hydrochloric acid solution prepared with distilled water, then put it in the thermostat and got removed after 30 min-48 h incubation.

### BENEFICIAL EFFECTS

The advantages of nano pores configuration for drug release used in drug-eluting device are as follows:

1. The present device body does not contain polymer, therefore the risk of future thrombus formation was lowered compared to implanting the drug carried by polymer in prior art.

2. Compared with micron pores, visible pores or drug storage, the nano pores have no effect on the mechanical properties of the device body. The animal experiments show that the safety and efficacy are no less than those of the polymer drug-eluting device used in the prior art, and sometimes are even higher than those in the prior art.

In view of the expected use of the stent, and ensuring the compatibility with the human body as good as possible, animal implanting experiment employs healthy pygmy pig which is the most similar animal model to human, to evaluate the property of stents inside the body. All stents are positioned into anterior descending branch and circumflex artery branch of coronary artery in healthy pygmy pig with the ratio of stent/artery at 1.1-1.25:1. Angiography are performed for all stents and some of stents are observed by intravascular ultrasound (IVUS) 28 days after implant, to figure out the situation about intimal hyperplasia and restenosis. The statistical results of QCA analysis of three kinds of stents after 28 days implantation are shown in the following table.

| | Stent kind | | |
|---|---|---|---|
| | H-S(12) | Pt(12) | N-S(12) |
| Average value of | 1.35 | 0.8 | 0.6 |
| luminal loss | | | |
| (mm) | | | |
| Average degree | 45 | 30 | 25 |
| of renarrowing | | | |
| (%) | | | |
| Restenosis | 45.46 | 16.67 | 8.33 |
| rate(%) | | | |

In the table, H-S represents stainless steel bare stent; Pt presents the polymer stent carrying rapamycin, the concentration of the rapamycin is 1.4 μg/mm2; N-S presents nanopore stent carrying rapamycin, the concentration of rapamycin is 1.4 μg/mm2;

The results of angiography and IVUS for all experimental pigs after 28 days demonstrated that both non-polymer nanopore drug-eluting stent and polymer drug-eluting stent show better effects than stainless steel bare stent on stent restenosis rate and luminal loss. Both the restenosis rate and luminal loss for bare stent are higher than those of drug-eluting stent, both restenosis rate and luminal loss for nanopore drug stent are slightly lower than those of polymer drug stent. The results indicated that the safety and the efficacy of lowering restenosis rate for the nanopores drug-eluting stent are no less than those of polymer drug stent with carrier.

As shown in FIG. 3, square point-line represents nanopore drug release curve, circular point line presents polymer drug release curve. Comparing to polymer carrying stent, the release rate of the nanopore stent of the present invention is faster during the first two days, but has no significant difference in general release trend. However, a little drug residue exists after 28 days, which ensures the continuance of the drug treatment.

3. The physical properties and supporting ability of the device body will not decrease, which could control the drug release rate efficiently, and decrease the restenosis ratio significantly after operation.

4. The present nanopore configuration can be widely used in medical devices with drug-eluting function. Specifically, when used in vascular stent, the nanopore configuration has perfect effect on vascular diseases treatment and vascular restenosis prevention.

5. The nanopores and the drug in nanopores are prepared in the device body raw material, without obvious interface, and the formation of the nanopores can be controlled easily.

6. There is no need to prepare an extra layer on device body to carry drug, which simplifies the preparation process, shortens the production period and reduces the production cost.

### DESCRIPTION OF DRAWINGS

FIG. 1 is the cross sectional diagram of drug release configuration of the current art using polymer carrying drug;

FIG. 2 is the cross sectional diagram of drug release configuration whose pores were prepared by laser in the current art;

FIG. 3 is the drug release curve of the present invention;

FIG. 4 is the cross sectional diagram of release configuration of the single-sized nanopores of the present invention, prepared in the device raw material;

FIG. 5 is the cross sectional diagram of release configuration of double sized (big-sized and small-sized) nanopores of the present invention, prepared in the device raw material;

FIG. 6 is the cross sectional diagram of release configuration of double sized (deep and shallow) nanopores of the present invention, prepared in the device raw material;

FIG. 7 is the cross sectional diagram of release configuration of multiple sized (triple or more sized) nanopores of the present invention, prepared in the device raw material;

FIG. 8 is a statistical distribution curve of the drug release configuration of single sized nanopores of the present invention, prepared in the device raw material directly;

FIG. 9 is a statistical distribution curve of the drug release configuration of multiple sized nanopores of the present invention, prepared in the device raw material directly;

FIG. 10 is a flow chart for the process of the present invention; and

FIG. 11 is the diagram of the anode pulse equipment of the present invention.

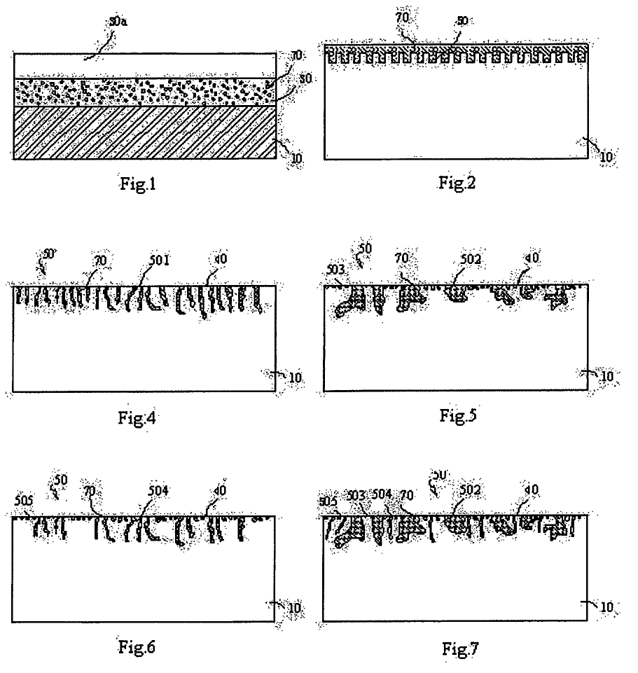

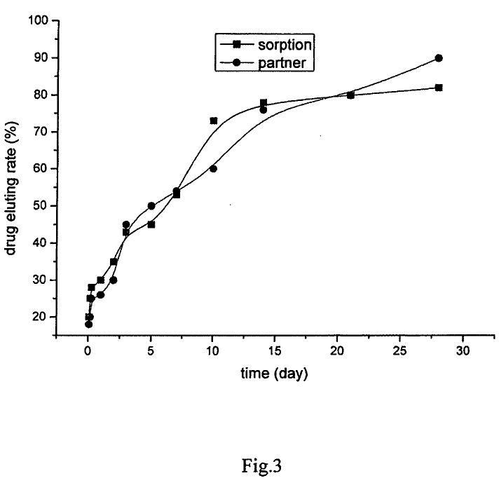

### DETAILED EMBODIMENT

Hereinafter, one of the embodiments of the nanoporous configuration for drug release used in the drug-eluting device and its preparation of the present invention will be described in detail with reference to accompanying drawings, which does not limit the claim scope of the present invention.

A nanoporous configuration for drug release used in drug-eluting device as shown in FIG. 4, mainly comprises device body 10, active drug 70, pores 50, membrane layer 40 etc. The said Pores 50 are a large number of nanopores. The nanopores do not mean that the diameter must be less than 100 nm strictly, in the present invention the pores with diameter less than or more than 1 nm are all named nanopores, which in details, means the pores with diameter between 1 μm and 1 nm. Nanopores 50 can be prepared in the raw material of device body 10 by chemical or physical method, such as corrosion, anode oxidation, micro-arc oxidation, micro-arc nitridation or the combination of these methods. No layer exists between nanopores 50 and device body 10, and the nanopores 50 could be tank or pore configuration for carrying drug. The said device body 10 could comprise an external membrane 40 or not; nanopores 50 could be single sized distribution, it is said that, the nanopores 50 have uniform size and the active drug 70 is loaded in each uniform sized nanopores 501 or adhered to the surface of device body 10.

As shown in FIG. 5, nonuniformly distributed double sized nanopores 50, or n double sized distributed nanopores 50 with twice the average diameter, can be prepared directly in the raw material of device body 10. Double sized nanopores 50 include two different diameter pores, large nanopores 502 and small nanopores 503. Active drug is loaded in every large nanopores 502 and small nanopores 503 or adhered to the surface of device body 10.

As shown in FIG. 6, nonuniformly distributed double sized nanopores 50, or n (n) double sized distributed nanopores 50 with twice the average value of depth, can be prepared directly in the raw material of device body 10. Double sized nanopores 50 include two different depth pores, deep nanopores 504 and shallow nanopores 505. Active drug is loaded in each deep nanopores 504 and shallow nanopores 505 or adhered to the surface of device body 10.

As shown in FIG. 7, nonuniformly distributed multiple sized nanopores 50, or n (n) multiple sized distributed nanopores 50 with three times the average diameter and depth, can be prepared directly in the raw material of device body 10. Multiple sized nanopores 50 include three or more different diameter or depth pores, large nanopores 502, small nanopores 503, deep nanopores 504 and shallow nanopores 505. Active drug is loaded in each large nanopores 502 and/or small nanopores 503 and/or deep nanopores 504 and shallow nanopores 505 or adhered to the surface of device body 10.

The single sized nanopores 50 can be any one of the uniform sized nanopores 501, large nanopores 502, small nanopores 503, deep nanopores 504, shallow nanopores 505.

The uniform sized nanopores 501, large nanopores 502, small nanopores 503, deep nanopores 504, shallow nanopores 505 can be open pores, half-open pores, closed pores, independent pores, interconnected pores, inter-embedded pores or nested pores as well as small pores existing in big pores, which can be chosen by drug dosage or medical devices.

The active drug in nanopores 50 or adhered to surface of device body 10 includes one or more substances such as pharmacotherapy agent, vector for gene therapy, bioactive substance and combinations thereof.

The pharmacotherapy agent of the present invention includes but is not limited to the following one or more substance: heparin, aspirin, hirudin, colchicine, antiplatelet GPIIb/IIIa receptor antagonist, Methotrexate, purines, miazines, alkaloid, Epothilone, Tripterygium Wilfordii series compound, antibotics, hormone, antibody drug for cancer treatment, cyclosporin, tacrolimus, homologues (FK506), 15-deoxyspergualin, Mycophenolate Mofetil(MMF), rapamycin, derivatives, FR 900520, FR 900523, NK 86-1086, daclizumab, valeramide (depsidomycin), kanglemycin C, spergualin, prodigiosin25-c, tranilast, myriocin, FR 651814, SDZ214-104, cyclosporinc, bredinin, mycophenolic acid, Brefeldin A, WS9482, glucocorticosteroid, tirofiban, abciximab, eptifibatide, paclitaxel, actinomycin-D, As₂O₃, 17 β-estradiol etc.

The vector for gene therapy includes but is not limited to one or more substance selected from: cell, virus, DNA, RNA, virus vector, non-virus vector.

The said bioactive substance includes but is not limited to one or more substances selected from: cell, yeast, bacteria, protein, peptide and hormone.

The device body 10 of the present invention includes the medical devices used for drug release, such as stent, duct, guidewire, cardiac pacemaker, cardiac valve, surgical implant material, implanted hard tissue, and nonmetal medical devices employing ceramic, organic polymer, inorganics, metal oxide as basic material. The stents are balloon expand stents, self expanding stents, vascular stents, non-vascular stents or stents employing medical stainless steel with good biocompatibility, nickel-titanium shape memory alloy, cobalt alloy, pure titanium, titanium alloy and tantalum, titanium alloy, or gold as basic material, and wire braided, pipe laser cut, mold casted, and welded stents.

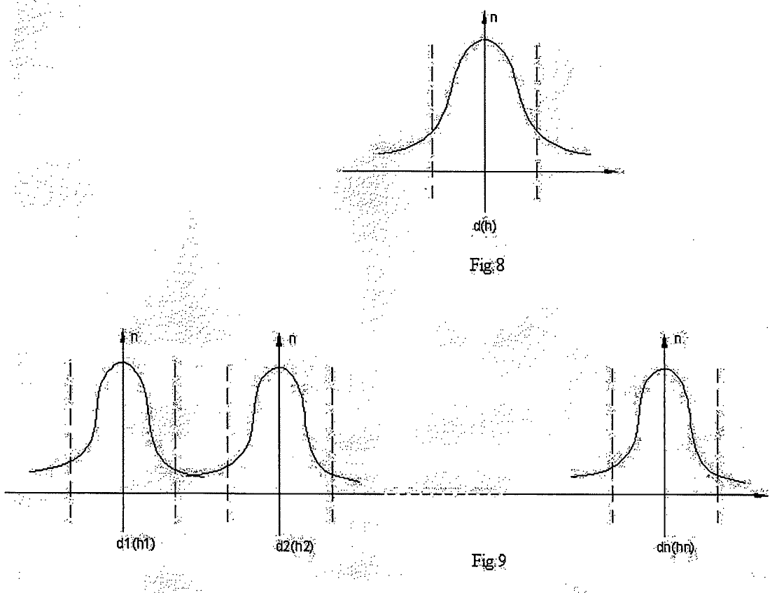

As shown in FIG. 8 and FIG. 9, the pores can be in any shape. Pore diameter d is the effective diameter of the pore, which means the diameter is obtained by converting any shape pore to an equivalent diameter circle pore under some geometric rules; the pore depth h is the distance from the bottom of the pore to the base surface of coating layer; the said size distribution is a statistical model which can describe the sizes of the pores, including pore diameter d and pore depth h distribution statistical model as the pores sizes would be not equal, but distributed under some statistical rules; the average size means the average value of two or more size in statistics, as well as the statistical average value of pore diameters or depths; average value of the diameter d and depth h of the said nanopores can be allocated in the range of 1 nm-500 μm.

The nanopores in FIG. 8 are pores with one average size, as the distribution of pores could be described by single distribution rule.

The nanopores in FIG. 9 have two or more sizes. These pores have two or n average sizes, when n=2, mean double sized pores, when n>2, mean multiple sized pores, as well as the gathering of the pores that need n≧2 distribution rules to describe the size of diameters d or depths h of the pores.

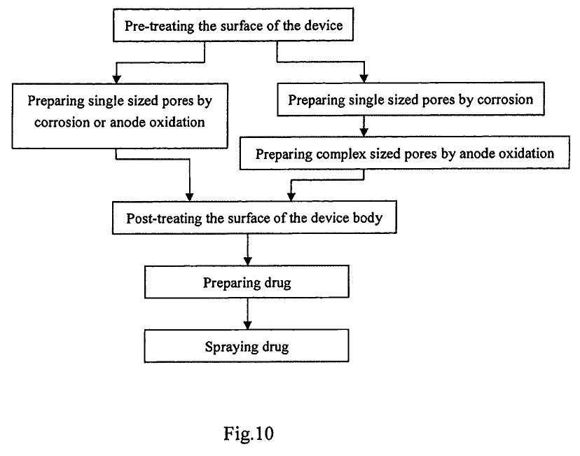

As shown in FIG. 10, a preparation method of a nanoporous configuration for drug release used in drug-eluting device, includes following process: \textcircled{1} Pre-treating the surface of the device body; \textcircled{2} Preparing pore a, b; \textcircled{3} Post-treating the surface of the device body; \textcircled{4} Preparing drug; \textcircled{5} Spraying drug etc. wherein:

\textcircled{1} Pre-treating the surface of the device body: employing ultrasonic waves to clean the impurities on the device body surface, for instance, using analytically pure acetone with concentration of 99.5% or medical grade alcohol with concentration of 75% to clean the bare stainless steel stent body material under ultrasonic wave of 28-100 khz wave for 5-15 min to remove the impurities on the surface of the device body, then putting the clean body material in dryer under 30-40° C. for 30˜60 min, after that, get out for use whereafter;

\textcircled{2} The steps of preparing pore a, b, includes preparing single sized nanonpores 50 and preparing multiple sized nanon complex pores 50:

When preparing single sized nanopores, nanopores 50 are prepared directly on raw materials of device body 10 by acid corrosion or anode oxidation.

Preparing pores by acid corrosion by immersing the device body materials in corrosion solution at 0-100° C., wherein hydrochloric acid with concentration of 1-38%, or mixed acid solution with 1-38% hydrochloric and 1-98% sulfuric acid, or 1-30% hydrofluoric acid, or the above three acids mixed in any concentration is preferred as the corrosion solution. The corrosion time limits to 1 min-480 h according to different concentration and temperature, thereafter the nanopores with diameters around 400 nm are formed on the surface of the body materials.

When preparing multiple sized nanon complex pores, firstly employing acid corrosion to prepare single sized nanopores, secondly preparing multiple sized complex nanopores 50 by anode oxidation or micro-arc oxidation combined with micro-arc nitridation. When employing anode pulse device or other pulsed power to perform anode oxidation, hydrochloric acid with concentration of 1-38% or sulfuric acid with concentration of 1-98% is preferably chosed for the electrolyte, and electric current is 0.01-0.1 A, frequency is 25-3000 Hz, time is 1-20 min.

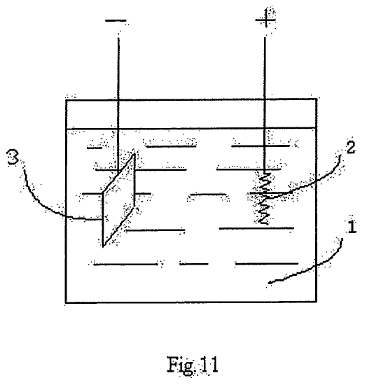

As shown in FIG. 11, when employing device body 10 as anode connecting to positive electrode of pulsed power, titanium flack 3 as cathode connecting to the negative electrode of pulsed power, putting stent 2 and titanium flack 3 into 20% hydrochloric acid 1 synchronously, under current at 0.1 A, frequency at 1667 Hz for 5 min, hereafter, nanopores 50 with complex configuration is prepared on the surface of the device body.

\textcircled{3} Post-treating the surface of the device body: cleaning the body material under ultrasonic wave for 5-15 min in analytically pure acetone with concentration of 99.5% sequentially in distilled water; putting the clean body material in dryer at 30-40° C. for 30-60 min, then removing out for use; or immersing the body material in the hydrochloric acid solution with concentration of 1˜38% prepared with distilled water, then putting it into the thermostat around 20° C. for 30 min-48 h, then getting it out.

\textcircled{4} Preparing drug: preparing sufficiently dissolved organic solution containing 0.01-10% (wt.) active drug (70); the ratio of the said active drug (70) to organic solution is 1:10-1:10000 in weight;

\textcircled{5} Spraying drug: fixing the device body on spraying machine, and spraying the above said prepared active drug (70) on the body material uniformly.

### PRACTICAL APPLICABILITY

The nanoporous configuration for drug release used in drug-eluting device could be used in various drug stents in medical devices, including blood vessel stents, esophagus stents, trachea stents etc.; implanted hard tissue with coating drugs, such as coax arthrosis, thigh arthrosis, cardiac valve and so on.

1. A nanoporous drug-eluting device that comprises a device body having a plurality of nanopores, one or more active drugs positioned in said pores or adhered to said device body, wherein said nanopores are single sized or two sized or multiple sized nanopores, having a uniform size distribution or two or more nonuniform size distributions in diameter or depth.

2. The nanoporous device according to claim 1, wherein the average value of pore diameter d and pore depth h of said nanopores is 1 nm-500 μm.

3. The nanoporous device according to claim 1, wherein said device body further comprises an external membrane layer.

4. The nanoporous device according to claim 1, wherein said single sized nanopores are uniform sized nanopores, large nanopores, small nanopores, deep nanopores, or shallow nanopores.

5. The nanoporous device according to claim 1, wherein said two sized nanopores include two different diameter pores, or two different depth pores, deep nanopores and shallow nanopores and active drug is loaded in each of said pores.

6. The nanoporous device according to claim 1, wherein said multiple sized nanopores include large nanopores, small nanopores, deep nanopores, shallow nanopores in three or more different pore diameters or depths; and active drug is loaded in said large nanopores and/or small nanopores and/or deep nanopores and/or shallow nanopores.

7. The said nanoporous device according to claim 3, wherein said uniform sized nanopores, large nanopores, small nanopores, deep nanopores, and shallow nanopores are open pores, half-open pores, closed pores, independent pores, interconnected pores, inter-embedded pores, nested pores, or small pores existing in big pores.

8. The nanoporous device according to claim 1, wherein said active drugs are selected from the group consisting of pharmacotherapy agents, vectors for gene therapy, and bioactive substances.

9. The nanoporous device according to claim 8, wherein said pharmacotherapy agents are selected from the group consisting of heparin, aspirin, hirudin, colchicine, antiplatelet GPIIb/IIIa receptor antagonist, Methotrexate, purine, miazine, alkaloid and Epothilone, Tripterygium Wilfordii series compound, antibotics, hormone, antibody drug for cancer treatment, cyclosporin, tacrolimus (FK506) and its homologues, 15-deoxyspergualin, Mycophenolate Mofetil (MMF), Rapamycin and its derivatives, FR 900520, FR 900523, NK 86-1086, daclizumab, valeramide (depsidomycin), kanglemycin C, spergualin, 25c(prodigiosin25-c), tranilast, myriocin, FR 651814, SDZ214-104, cyclosporinC, bredinin, mycophenolic acid, Brefeldin A, WS9482, glucocorticosteroid, tirofiban, abciximab, eptifibatide, paclitaxel, actinomycin-D, As₂O₃, 17 β-estradiol.

10. The nanoporous device according to claim 8, wherein said vectors for gene therapy are selected from the group consisting of cell, virus, DNA, RNA, virus vectors, and non-virus vectors.

11. The nanoporous device according to claim 8, wherein said bioactive substances are selected from the group consisting of cell, yeast, bacteria, protein, peptide and hormone.

12. The nanoporous device according to claim 1, wherein said device body includes stents, duct, guidewire, cardiac pacemaker, cardiac valve, surgical implant material, implanted hard tissue, and nonmetal medical devices employed ceramic, organic polymer, inorganics, metal oxide as basic material; wherein said stent is a balloon expanded stent, self expanding stent, vascular stent, non-vascular stent, and wherein said stent employ medical stainless steel with good biocompatibility, nickel-titanium shape memory alloy, cobalt alloy, pure titanium, titanium alloy, or tantalum titanium alloy, or gold as a basic material, and wherein said stent is wire braided, pipe laser cut, mold casted, or welded.

13. A method of a making a nonporous device for drug release, said method comprising the steps of:
\textcircled{1} pretreating the surface of a device body;
\textcircled{2} preparing pores by acid corrosion, or directly making single sized nanopores on material of device body by anode oxidation; or making single sized nanopores by acid corrosion on material of device body firstly, and then making multiple sized complex nanopores by anode oxidation or micro-arc oxidation combined with micro-arc nitridation;
\textcircled{3} post-treating the surface of the device body;
\textcircled{4} preparing an organic solution containing 0.01-10% (wt.) dissolved active drug; whereby the ratio of said active drug to organic solution is 1:10-1:10000 by weight; and
\textcircled{5} fixing said device body to a spraying machine, and spraying said active drug solution on said body material uniformly.

14. The method according to claim 13, wherein the step of preparing pores by acid corrosion comprises immersing the device body materials in corrosion solution at 0-100° C., wherein the said corrosion solution is preferred to be hydrochloric acid with concentration of 1-38%, or mixed acid solution with 1-38% hydrochloric and 1-98% sulfuric acid, or 1-30% hydrofluoric acid, or the above said three acids mixed in any concentration, and controlling the corrosion time in 1 min-480 h whereby the uniformly sized nanopores are formed.

15. The method according to claim 13, wherein the step of anode oxidation includes employing device body material as an anode connecting to a positive electrode of pulsed power, titanium flake as a cathode connecting to a negative electrode of pulsed power, depositing the stent and titanium flake in hydrochloric acid simultaneously, wherein the electrolyte is preferred to be hydrochloric acid with concentration of 1-38% or sulfuric acid with concentration of 1-98%, the electric current is 0.01-0.1 A, frequency is 25-3000 Hz, and time is 1-20 min.

16. The method according to claim 13, wherein the step of pre-treating the surface of a device body includes cleaning the impurities on device body surface by acetone or alcohol solvent under sonication.

17. The method according to claim 13, wherein post-treating the surface of the device body includes cleaning the device body through the above treatment by acetone and distilled water sequentially under ultrasonic condition, drying the clean device body material in dryer or preparing hydrochloric acid solution with distilled water, immersing the body material in it, then putting in thermostat and getting out after 30 min-48 h.
